(19) United States
(12) Patent Application Publication
(10) Pub. No.: US 2026/0124234 A1
(43) Pub. Date: May 7, 2026

(54) METHOD FOR TREATING A NEUROLOGICAL DISORDER IN AN ANIMAL

(71) Applicants: Research Foundation of the City University of New York, New York, NY (US); Centre national de la recherche scientifique, Paris (FR)

(72) Inventors: John H. Martin, New York, NY (US); Fatiha Nothias-Chahir, Paris (FR)

(21) Appl. No.: 19/353,360

(22) Filed: Oct. 8, 2025

Related U.S. Application Data
(60) Provisional application No. 63/704,770, filed on Oct. 8, 2024.
(60) Provisional application No. 63/894,367, filed on Oct. 6, 2025.

(51) Int. Cl. A61K 31/722; A61K 9/06; A61N 1/20; A61N 1/36; A61P 25/28

(52) U.S. Cl. CPC A61K 31/722; A61K 9/06; A61N 1/20; A61N 1/3606; A61P 25/28

(57) ABSTRACT

A method for treating a neurological disorder in an animal. A chitosan Fragmented Physical Hydrogel Suspension (C_{fphs}) is administered to the animal at a site of spinal cord injury (SCI). Thereafter both (1) cerebral cortex neuromodulation and (2) spinal cord direct current stimulation (tsDCS) are performed on the animal.

### CROSS-REFERENCE TO RELATED APPLICATIONS

This application claims priority to, and is a non-provisional of, U.S. Patent Applications 63/704,770 (filed Oct. 8, 2024) and 63/894,367 (filed Oct. 6, 2025), the entirety of which is incorporated herein by reference.

### STATEMENT OF FEDERALLY SPONSORED RESEARCH OR DEVELOPMENT

This invention was made with Government support under grant number 1R21NS116550 awarded by the National Institutes of Health. The government has certain rights in the invention.

### BACKGROUND OF THE INVENTION

The subject matter disclosed herein relates to treatments for neurological damage and to treatments for spinal cord injury (SCI) in particular.

SCI produces extensive damage at the trauma site, leading to neural cell loss, axon injury, and cystic cavitation. Combined injury site damage and interruption of ascending, descending and local axons produces motor, sensory, and autonomic impairments. Several factors contribute to the complexity of SCI physiopathology including breakdown of the blood-spinal cord barrier, inflammation spread, free radicals and glutamate excitotoxicity—limiting spontaneous repair. The initial trauma leads to secondary structural damage that spreads to adjacent spinal segments, characterized by axon dieback, demyelination and neural cell death. This is largely due to persistent inflammation enlarging the lesion caudally and rostrally. The developing cavities become walled off by the astrocytic scar, creating a physical-chemical barrier to axon growth, in addition to potent growth inhibitory factors (e.g., myelin debris and chondroitin sulfate proteoglycans). The progressive damage at the injury site expands the loss of neural connections with associated functional consequences. Although a variety of treatment methods have been tested, they remain limited in their ability to achieve synergistically both tissue repair and promoting projections for adequate neuronal connectivity. Alternative treatment mods are therefore desirable.

The discussion above is merely provided for general background information and is not intended to be used as an aid in determining the scope of the claimed subject matter.

### SUMMARY

A method for treating a neurological disorder in an animal. A chitosan Fragmented Physical Hydrogel Suspension (C_{fphs}) is administered to the animal at a site of spinal cord injury (SCI). Thereafter both (1) cerebral cortex neuromodulation and (2) spinal cord direct current stimulation (tsDCS) are performed on the animal.

An advantage that may be realized in the practice of some disclosed embodiments of the method is that corticospinal tract (CST) gray matter axon density is significantly enhanced. This only occurred when the combined C_{fphs} and neuromodulation treatment is performed. Furthermore, when only the neuromodulation treatment is performed, only 12.5% axon growth was found below the lesion (see Exp Neurol. 2017 November; 297: 179-189). In contrast, when the combined C_{fphs} and neuromodulation treatment is performed, axon growth increases two-fold.

In a first embodiment, a method for treating a neurological disorder in an animal is provided. The method comprising steps of: administering chitosan Fragmented Physical Hydrogel Suspension (C_{fphs}) to the animal at a site of spinal cord injury (SCI); performing both (1) cerebral cortex neuromodulation and (2) spinal cord direct current stimulation (tsDCS) on the animal for at least 10 minutes per day; and repeating the step of performing for at least 10 days.

In a second embodiment, a method for treating a neurological disorder in an animal is provided. The methods comprising steps of: administering chitosan Fragmented Physical Hydrogel Suspension (C_{fphs}) to the animal at a site of spinal cord injury (SCI), wherein the C_{fphs} comprises chitosan hydrogel microparticles with a median size d50, obtained from a number distribution, of from 1 to 500 μm, a degree of acetylation of less than or equal to 20%, wherein chitosan in the hydrogel microparticles is from 0.25 to 5% concentration by weight, based on the total weight of the C_{fphs}; performing both (1) motor cortex (MCX) neuromodulation and (2) spinal cord direct current stimulation (tsDCS) on the animal for at least 10 minutes per day; and repeating the step of performing for at least 10 days.

This brief description of the invention is intended only to provide a brief overview of subject matter disclosed herein according to one or more illustrative embodiments and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a timeline of an experimental procedure. All animals were prepared with anterograde tracers and epidural electrodes in primary motor cortex (MCX) prior to injury. All animals received a moderate (200 kdyn) C4 midline contusion (SCI). Animals treated with the biomaterial C_{fphs} were injected on day 3 post-injury. Animals treated with neuromodulation were administered dual MCX-spinal electrical stimulation sessions on 10 sequential days (gray shading). The experiments were terminated at 8 weeks for lesion pathology and corticospinal axon complexity analyses.

FIGS. 2A-2E are fluorescent images showing SCI cavitation and scarring. FIG. 2A depicts SCI only (scale 500 km). Typical cavitation (asterisk) surrounded by a dense band of gliosis, viewed at the lesion epicenter. FIG. 2B (scale 500 μm) depicts SCI+C_{fphs}. Example of reduced lesion cavity and gliosis in an SCI animal treated with C_{fphs} injection. FIG. 2C (scale 500 km) shows the remodeled injury site (yellow asterisks) is abundant with fibers (Tuj1) and cells. FIG. 2D (scale 500 km) shows the results of staining with DAPI. The lower portion of the resolved lesion has a lower cell density, with small gaps that may reflect the presence of non-biodegraded C_{fphs}. FIG. 2E (scale 50 km) shows the resulting GFAP and Tuj1 staining from the rat that had received C_{fphs} injection (approximate image location corresponds to white square in FIG. 2B and FIG. 2C). Note spatial alignment between Tuj1 fibers and astrocytic processes (FIG. 2E). FIG. 2F is a graph that quantifies the cavitation area (mean±SE; asterisks indicate significance). The inset shows a schematic of the cavity/region of resolved cavity (black fill) and the GFAP regions of interest (ROI) (yellow fill). FIG. 2G is a graph that quantifies glial scar density (mean±SE; asterisks indicate significance).

FIG. 3A-3C (FIG. 3A shows scale bar; 500 μm) are images showing localization of CST axons at rostral injury site. Left panel of each figure shows CST axons in the dorsal column at the rostral injury site, and axons in the adjoining gray matter and laterally. Squares indicate location of the insets. Note arrows in B pointing to CST axons in the injury core. Right panel shows overlay of CST axons (inverted for visualization) and PDGFRB staining. Insets show PDFFRB staining alone. Landmarks (asterisks) used for image registration. FIG. 3A shows SCI only. FIG. 3B shows SCI+C_{fphs}. FIG. 3C shows SCI+C_{fphs}+NM.

FIGS. 4A-4C depict CST axon dieback relative to peak GFAP staining. FIG. 4A (scale 300 μm) shows SCI only. FIG. 4B (scale 300 μm) shows SCI+C_{fphs}+NM CST (black) and GFAP (red) imaging channels overlaid to show localization of the axon in the remodeled tissue (right, caudal). FIG. 4C shows plots GFAP staining intensity relative to baseline levels in register with the anatomical sections above. GFAP peak intensity (dotted line in FIG. 4C) denotes the lesion margin and is aligned with arrows in FIG. 4A and FIG. 4B. FIG. 4D shows distribution of CST axon dieback in the rostral dorsal columns. The shading indicates the regions examined caudal to the lesion margin. FIG. 4E and FIG. 4F are graphs showing a comparison of axon dieback at 1.2 mm and 0.0 mm from the lesion margin, respectively. FIG. 4G is a graph showing rate of axon dieback. Numbers of animals: In FIG. 4E to FIG. 4G SCI, N=4; C_{fphs}, N=5; C_{fphs}+NM, N=6. In FIG. 4F SCI, N=4; C_{fphs}, N=5; C_{fphs}+NM, N=6. In FIG. 4H SCI, N=3; C_{fphs}, N=5; C_{fphs}+NM, N=5.

FIGS. 5A-5C are graphs depicting CST gray matter axon density. FIG. 5A shows CST sprouting rostral to the injury at C3. The total normalized length of CST axons within the ROI was determined. FIG. 5B shows CST sprouting at the rostral lesion margin. FIG. 5C shows CST sprouting immediately caudal to the injury at C5. The inset in FIG. 4A shows the ROIs for FIG. 5A to FIG. 5C. Number of animals for analyses: In FIG. 5A and FIG. 5B SCI, N=4; C_{fphs}, N=5; C_{fphs}+NM, N=5. In FIG. 5C SCI, N=4; C_{fphs}, N=5; C_{fphs}+NM, N=7.

FIG. 6A to FIG. 6D shows CST axon length, branching, and dorso-ventral distribution caudal to the lesion. FIG. 6A shows examples of CST axon terminals (AAV-mCherry, red) in the gray matter in a C_{fphs}+NM rat. Left, low magnification view showing the lesion in relation to two CST terminal examples (arrows). Center, right. Axon arbors at higher magnification (arrows in FIG. 6A). FIG. 6B shows CST axon mean length±SEM. C. CST axon branching±SEM. Measurements are based on the number of branch points for each measured axon in FIG. 5C. The inset in FIG. 6B shows a schematic and the C5-C7 region of interest for analyses in FIG. 6B-6D. Axons were measured and normalized (see FIG. 6C) to the tracer efficacy factor as for FIGS. 6A-6C. FIG. 6D, the density of CST axons separated by zones in the dorsal and intermediate regions. The plots are overlaid onto a schematic of the spinal cord with contours to aid reference of the relative dorsoventral distribution. Numbers of animals for analysis: SCI only, N=4; C_{fphs}, N=5; C_{fphs}+NM, N=7. Calibrations: FIG. 6A. Left, 500 μm; Center and right, 100 μm. FIG. 6D 0.3 axon density.

### DETAILED DESCRIPTION OF THE INVENTION

This disclosure provides a method for promoting tissue repair and enhancing descending projections after injury or pathological events (e.g. spinal cord injury (SCI), traumatic brain injury (TBI), stroke, amyotrophic lateral sclerosis (ALS)). For tissue repair, an engineered chitosan hydrogel formulation (Chitosan Fragmented Physical Hydrogel Suspension; C_{fphs}, see Chedly et al., 2017, Biomaterials 138, 91-107; Soares et al., 2020, Rev. Neurol. (Paris) 176, 252-260) is administered. A combinatorial treatment of dual brain and spinal cord stimulation is then utilized to promote nervous tissue repair and neural connectivity after injury. The combinatorial treatment targets a particular brain circuit—the corticospinal tract. The treatment includes intermittent electrical theta burst stimulation from the origin area (distant from the lesion where the cell bodies of the neurons are located) of the fibers that project through the injured region. To further promote re-connectivity, the intermittent electrical theta burst stimulation is combined with local non-invasive transcutaneous direct stimulation (tsDCS) at the lesioned portion of the spinal cord. For example, the neuromodulation combines motor cortex intermittent electrical theta burst stimulation and spinal tsDCS.

For promoting descending projections, the corticospinal tract (CST) was targeted, the primary system controlling voluntary movement in humans and the system most responsible for loss of motor function after SCI. After injury, the CST is particularly vulnerable to axon dieback and is recalcitrant to injury-dependent growth. Cerebral cortex neuromodulation, such as motor cortex (MCX) neuromodulation promotes corticospinal tract (CST) structural plasticity, as well as long-term potentiation (LTP) at the CST-spinal interneuron synapse. In the disclosed method, MCX stimulation is combined with spinal cord direct current stimulation (tsDCS) to enhance motor evoked potential (MEP) responses by increasing spinal segmental excitability.

For example, intraspinal injection of C_{fphs} into an acute rat SCI modulate inflammation was used to achieve substantial lesion resolution compared with untreated SCI. C_{fphs} reduced the dense glial scar and renders the barrier between intact and lesioned tissue more permissive to invading cells and growing axons, allowing them to survive in newly formed and vascularized tissue, and prevent cavity formation at a chronic time point.

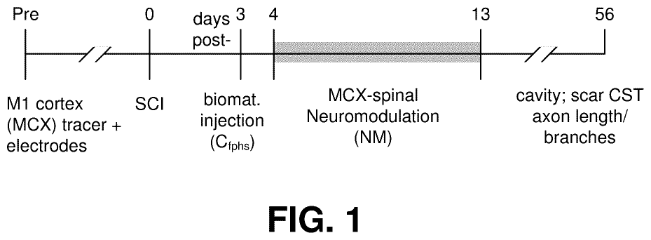

C_{fphs} was injected into the site of a 4th cervical segment spinal contusion during the acute period, followed by 10-days of MCX-tsDCS neuromodulation. See FIG. 1. In other embodiments, the C_{fphs} is injected within 60 days, within 56 days, within 30 days, within 20 days, within 10 days or within 5 days of injury. Herein, this combined neuromodulation treatment is referred to as NM. SCI (only) was compared with SCI+C_{fphs} and SCI+C_{fphs}+NM at a chronic time point. C_{fphs} alone or in combination with neuromodulation similarly abrogated cavitation and astrogliosis within the lesion site. C_{fphs} alone reduced CST axon dieback and prevented the associated loss of gray matter terminations rostral to SCI. CST axons were observed within remodeled injury site tissue. Only the combined C_{fphs} and neuromodulation treatment significantly enhanced CST gray matter axon density within the cervical gray matter, through activity-dependent axon sprouting rostral and caudal to the injury. The disclosed biomaterial-neuromodulation combinatorial strategy achieves significant lesion resolution and enhanced CST projections rostral and caudal to SCI.

C_{fphs} was prepared according to Chedly et al., 2017, Biomaterials 138, 91-107; Soares et al., 2020, Rev. Neurol. (Paris) 176, 252-260. Also see U.S. Pat. No. 9,623,044, the content of which is incorporated by reference. The combined MCX-tsDCS was performed according to Zareen et al., Exp. Neurol. 307, 133-144; Yang et al., 2019, Exp. Neurol. 320, 112962. The content of these documents is hereby incorporated by reference.

In one embodiment, the C_{fphs} comprises chitosan hydrogel microparticles with a median size d50, obtained from a number distribution, of from 1 to 500 μm, a degree of acetylation of less than or equal to 20% the concentration of chitosan in the hydrogel is from 0.25 to 5% by weight, based on the total weight of the hydrogel.

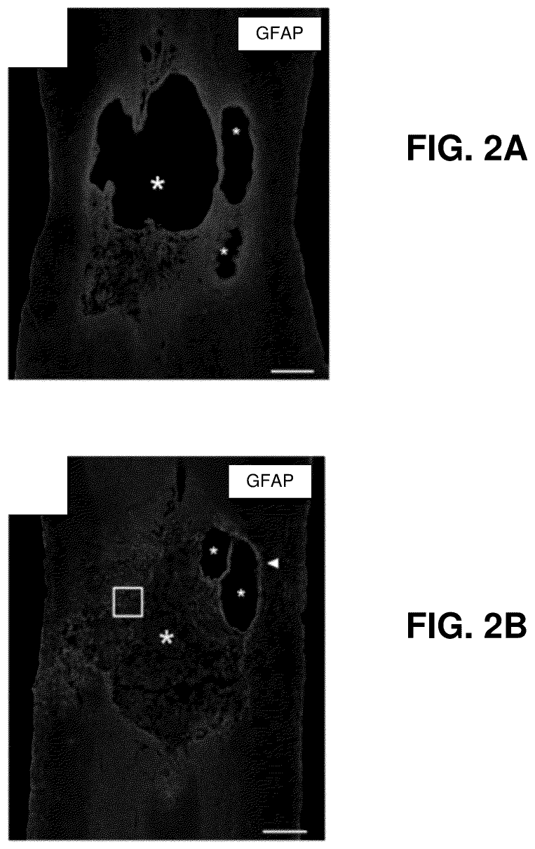

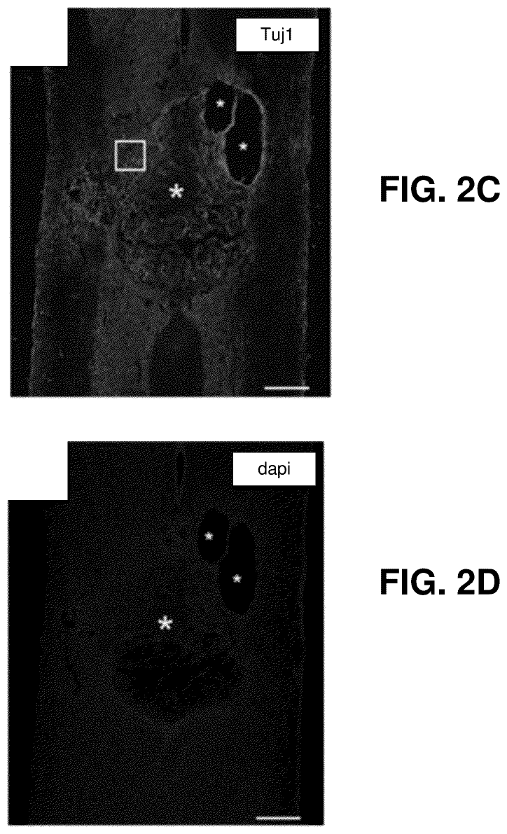

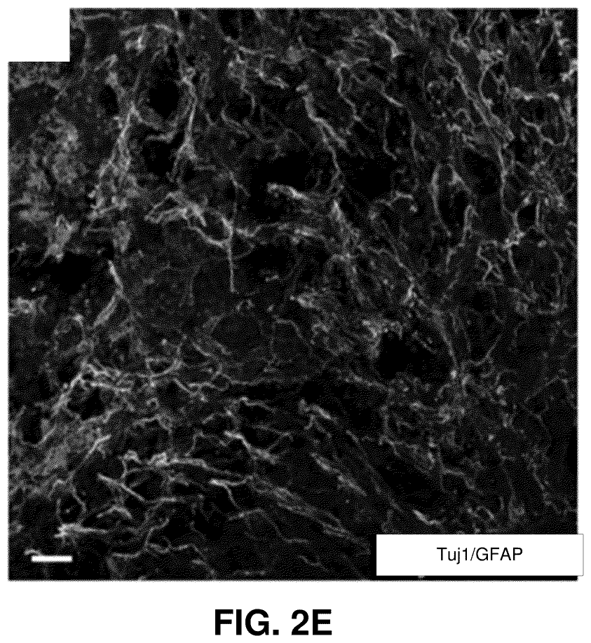

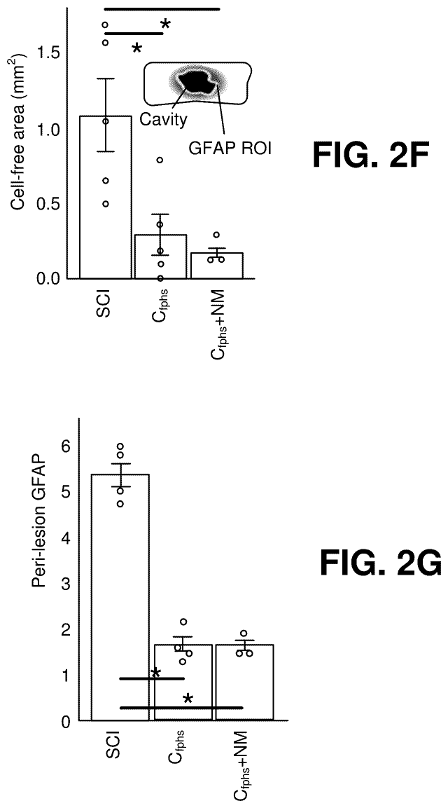

C_{fphs} abrogated the lesion cavity and the glial scar: At 8-weeks post injury, cervical midline contusion produces a large cavity at the lesion epicenter, typically with multiple smaller secondary cavities (asterisks; FIG. 2A). Surrounding each of the cavities is a ring of dense glial fibrillary acidic protein (GFAP) staining (FIG. 2A), similar to what was observed after thoracic dorsal hemisection (Chedly et al., 2017, Biomaterials 138, 91-107). These typical lesion characteristics were not observed in animals that received C_{fphs} injection. The main cavity was abrogated (FIG. 2B, yellow asterisk) and the lesion epicenter was filled with Tuj1+ axons (FIG. 2C) and cells (FIG. 2D, DAPI staining) also similar to thoracic dorsal hemisection (Chedly et al., 2017, Biomaterials 138, 91-107). In addition, GFAP in the remodeled tissue at the injury site was substantially reduced, especially the dense ring that is observed surrounding the cavity when C_{fphs} was not injected (FIG. 2A). Occasionally some GFAP-positive cells are located within the remodeled tissue of the lesion core (FIG. 2B, asterisk). This staining may correspond to GFAP expressing Schwann cells.

Schwann cells populate the injury core where they ensheathe or myelinate axons (Chedly et al., 2017, Biomaterials 138, 91-107). Maintenance of small ectopic (secondary) cavities with prominent GFAP staining (FIG. 2B-D, white asterisks) was occasionally observed, which may be due to incomplete filling by C_{fphs} and, consequently, the occurrence of a secondary lesion. Portions of the injury site were also observed where 4′,6-diamidino-2-phenylindole (DAPI) staining was focally lower in density. The gaps may reflect incompletely bio-degraded C_{fphs} at the 8-week time point. Importantly, these regions are not associated with dense GFAP staining (i.e., no strong reactive response by astrocytes) as in SCI only (FIG. 2A), which further demonstrate the compatibility of the biomaterial in contact with the central nervous tissue. Within the region of newly formed tissue at the injury center, Tuj1+axons are commonly aligned with astrocyte GFAP+ processes, especially near the interface with surrounding intact tissues (FIG. 2E), suggesting interactions that may assist local axon growth. After C_{fphs} injection, astrocytic delineation of the newly formed tissue is less prominent than in SCI only.

To evaluate the anatomical observations described above, cavity size and the intensity of GFAP staining was quantified, both because of their relevance to SCI pathophysiology and repair and that their characteristics are amenable to direct measurement. For the cavity, quantification (FIG. 2F; SCI, N=5; C_{fphs}, N=5; C_{fphs}+NM, N=5) revealed that C_{fphs} significantly reduced cavity size (KW=8.06, P=0.009) by 3.7-fold with C_{fphs} compared to SCI-only (P=0.024), as indicated by post hoc testing. Similarly, cavitation was also reduced in C_{fphs}+NM (P=0.009) with no further reduction compared to C_{fphs} only (P=0.7). For GFAP+ astrocytes, quantification showed that the dense staining surrounding the cavity in the SCI only group is significantly reduced in all C_{fphs} treated animals and delineating the newly formed tissue after contusion (SCI, N=5; C_{fphs}, N=5; C_{fphs}+NM, N=5) (KW=9.38, P=0.003). Post-hoc testing indicated that the astrocytic scar was reduced 3.2-fold in both C_{fphs} (P=0.007) and C_{fphs}+NM (P=0.009) compared to SCI (C_{fphs} same as C_{fphs}+NM, P=0.94). Thus, C_{fphs} produces significant reduction in cavitation and gliosis, with no further reduction with neuromodulation.

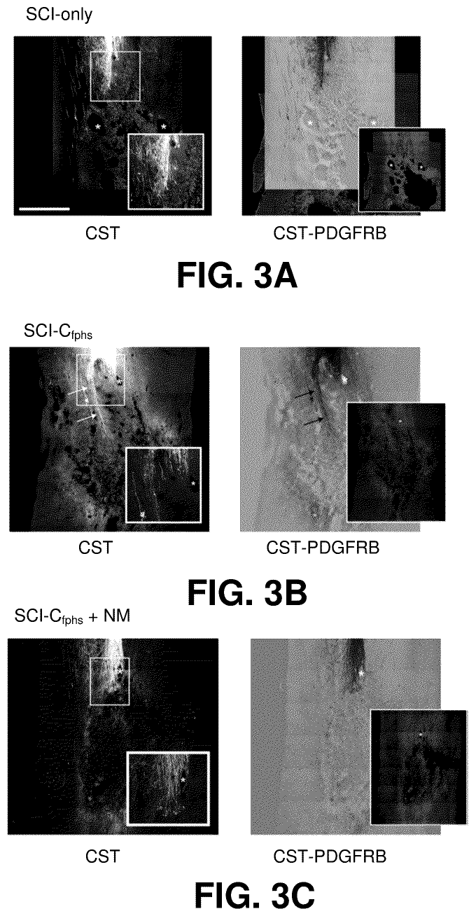

CST axons within the lesion site: To characterize the location of CST axons within the remodeled tissue of the injury site further, a platelet derived growth factor receptor beta (PDGFRB) staining was used. PDGFRB was used both because it stains mainly perivascular fibroblasts and pericytes, thus informing the extent of the scar in chronic lesion injury site, and that it allows CST axons to be localized within the remodeled injury site tissue (i.e., where the cavity would have been if C_{fphs} was not injected). PDGFRB also stains Schwann cells, which are present in large numbers in the newly formed tissue at the injury core (Chedly et al., 2017, Biomaterials 138, 91-107). For SCI only, CST labeling (FIGS. 3A-3C; left panel, CST alone; right panel, CST-PDGRFB image overlay) reveals axons extending close but not into the PDGFRB+ cavity margin. By contrast, animals receiving C_{fphs} alone (FIG. 3B) or with neuromodulation (FIG. 3C) show extension of axons into the newly formed tissue; even PDGFRB positive tissue. Axon extension is described further elsewhere in this disclosure on quantitative assessment of axon dieback. FIG. 3B (arrows) extension of CST axons into the PDGFRB positive remodeled tissue at the injury site. These results provide further support that C_{fphs} renders the injury site permissive to CST axon growth, and more generally revealed by Tuj1 staining. Importantly, while C_{fphs} abrogates the dense GFAP scar, it does not appear to mitigate PDGFRB staining; rather, it makes inhibitory regions more permissive to axon growth.

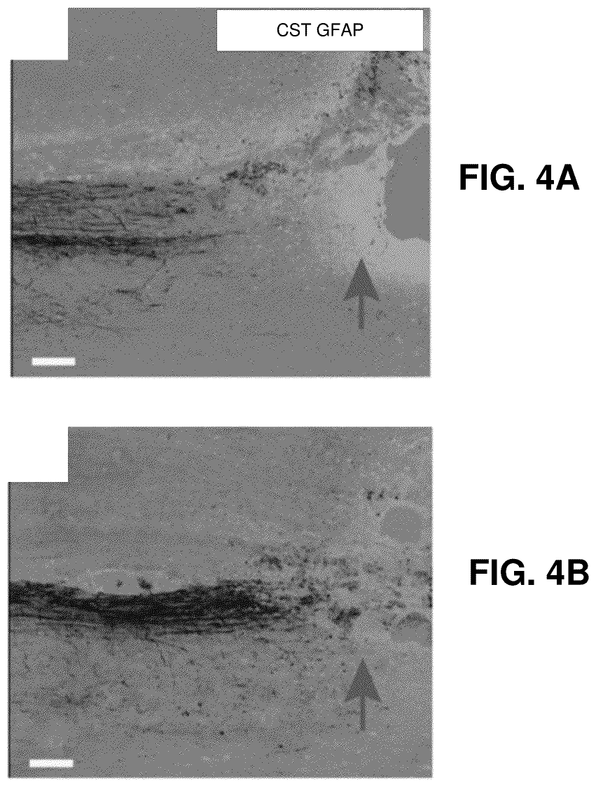

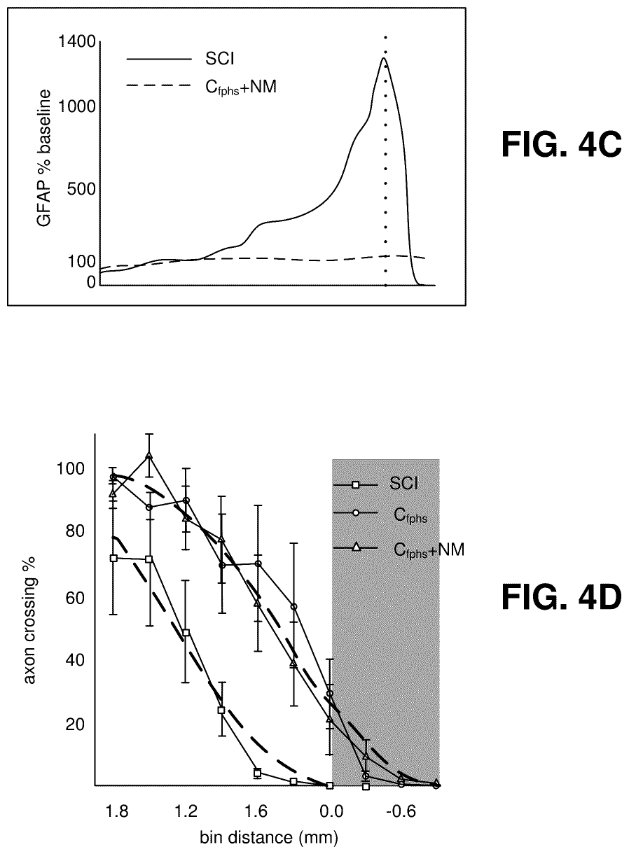

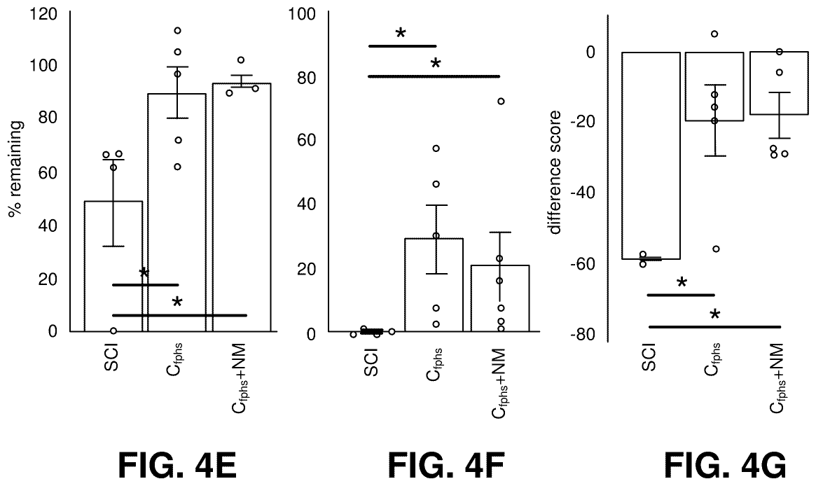

C_{fphs} reduced CST axon dieback: No labeled CST axons were observed in the dorsal column caudal to the injury confirming that the major CST pathway in the ventral dorsal columns was completely axotomized, similar to our prior studies following identical contusion parameters. The effects of C_{fphs} and C_{fphs}+NM on CST axon dieback in the dorsal columns after SCI were also examined (FIGS. 4A-4G). Location of the end of each CST axon was measured in relation to the peak GFAP staining intensity. CST axons (black) are superimposed on the GFAP channel (red) of the same section for representative SCI-only (FIG. 4A) and SCI+C_{fphs}+NM rats (FIG. 4B). Arrows mark the peak in measured GFAP staining (FIG. 4C). CST axons are typically observed to extend farther caudally, and into the injury site, exemplified in the C_{fphs}+NM case shown in FIG. 4B, compared to animals that did not receive C_{fphs}. Whereas no differences were noted in endbulb shape, prevalence, or size across the three groups, more collateral gray matter was observed branching in the C_{fphs} groups. The line plots (FIG. 4D) summarize the changes in axon dieback relative to plateau CST counts (measured farther rostrally). There is a caudal (right-ward) shift in the distribution of the C_{fphs} and C_{fphs}+NM groups relative to SCI only (solid lines plot raw data).

To quantify this difference in CST axon dieback further, axon counts were examined at two distances from the lesion rostral margin indicated by the peak in GFAP staining profile. At 1.2 mm (FIG. 4E), where there are no obvious signs of pathology and the GFAP analysis revealed a similar level of staining as in the intact tissue, there were significant increases in the number of remaining axons for the C_{fphs} groups compared with SCI-only (1.2 mm: KW=6.49, P=0.029, post-hoc SCI+C_{fphs}, P=0.023; SCI+C_{fphs}+NM, P=0.027, SCI+C_{fphs} same as SCI+C_{fphs}+NM, P=0.94; SCI-only, N=4; C_{fphs}, N=5; C_{fphs}+NM, N=5). A similar pattern of results was observed at the lesion margin (FIG. 4F; 0.0 mm), where dieback was reduced in the C_{fphs} groups compared with SCI (0.0 mm: KW=8.48, P=0.006, Post-hoc SCI+C_{fphs}, P=0.007; SCI+C_{fphs}+NM, P=0.015, SCI+C_{fphs} same as SCI+C_{fphs}+NM, P=0.68). To examine the rate of dieback for individual animals (FIG. 4G), a difference score was computed for the number of axons remaining from 1.2 mm to the midway point of the lesion margin (i.e., 0.6 mm from GFAP peak). There were group differences in the rate of dieback (KW=6.49, P=0.025). The dieback rate was 3-fold greater in the SCI-only group (58.82±0.73%) compared to SCI+C_{fphs} (19.76±9.99, P=0.017) and SCI+C_{fphs}+NM (18.32±6.43, P=0.029; SCI+C_{fphs} same as SCI+C_{fphs}+NM, P=0.81). Returning to the difference in the distributions of SCI-only (gray dashed line, FIG. 4D) and the C_{fphs} groups (combined average of C_{fphs} only and C_{fphs}+NM), these significant changes produce a caudal shift of the C_{fphs} groups of approximately 1000 μm (measured at 50% of total axon number in the dorsal column). These findings show substantial reductions in CST axon dieback and the rate of axon decline approaching the lesion after C_{fphs} injection, indicating the presence of CST axons significantly farther into the lesion site.

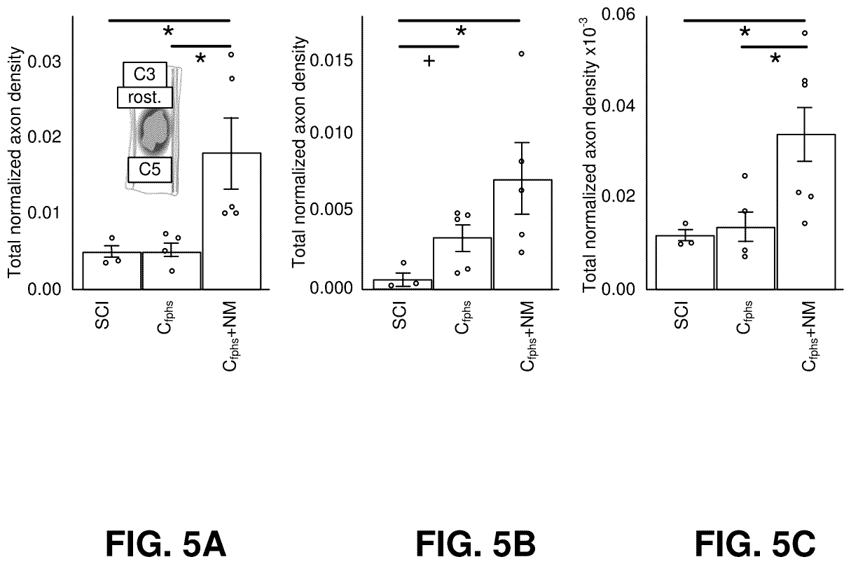

Changes in CST termination length at the rostral lesion border and Caudally: To determine how C_{fphs} and C_{fphs}+NM affects the length of CST axons in the rostral perilesional region, CST gray matter axon length was measured within rostral ROIs relative to the location of the peak GFAP labeling intensity. Variability in axon length and density measurements were controlled for due to the efficacy of tracer uptake based on total number of labeled CST axons at C2 (see Methods; section on Tracer efficacy normalization). For SCI-only rats, peak GFAP labeling corresponds to the thick dense band (FIG. 2A) and for the C_{fphs} groups, a local peak in the measured GFAP intensity, which corresponded to a faint band of staining that surrounded the newly formed tissue at the lesion site (FIG. 2B). CST density was measured approximately 1.5 mm rostral to peak GFAP, within the C3 gray matter lateral to the dorsal columns, where there normally are extensive CST collateral axon branches (FIG. 5A; see inset in A for ROI locations). There was approximately a 3.5-fold increase in axon density for C_{fphs}+NM over the SCI (P=0.008) and C_{fphs} (P=0.013) amounts (KW=9.103, P=0.002; SCI same as C_{fphs} P=0.26). The increase in the C_{fphs}+NM group at this rostral site is consistent with the known effect of dual neuromodulation on CST axon sprouting in intact rats and after cervical SCI.

At the region at peak GFAP immunostaining (i.e., the lesion site; 0 mm), there were group differences in axon density (FIG. 5B; KW=7.77, P=0.01). Post-hoc comparisons showed that the C_{fphs}+NM group had significantly elevated axon density compared with SCI-only (P=0.005). As used in this specification, an improvement is said to be significant if there is a p-value of at least 0.05 when compared to a control group. Whereas the C_{fphs} and C_{fphs}+NM groups did not differ (P=0.26), the effect of C_{fphs} compared with SCI was near significance (P=0.08). The findings suggest that C_{fphs} alone helps maintain CST gray matter axons at the rostral injury site and, with neuromodulation, produces activity-dependent CST axon sprouting.

Immediately caudal to the lesion at C5 (FIG. 5C), a bilateral area (about 2 mm² on average) was imaged and a significant group effect for axon density (KW=7.90, P=0.010) was found. There was a 2.5-fold increase in axon density with C_{fphs}+NM compared to SCI-only (P=0.018). CST density in the C_{fphs}+NM group was also higher than in the C_{fphs} group (P=0.02). There was no difference between SCI-only and C_{fphs} groups (P=0.86). These findings show that C_{fphs}+NM produces strong activity-dependent sprouting rostral and caudal to SCI.

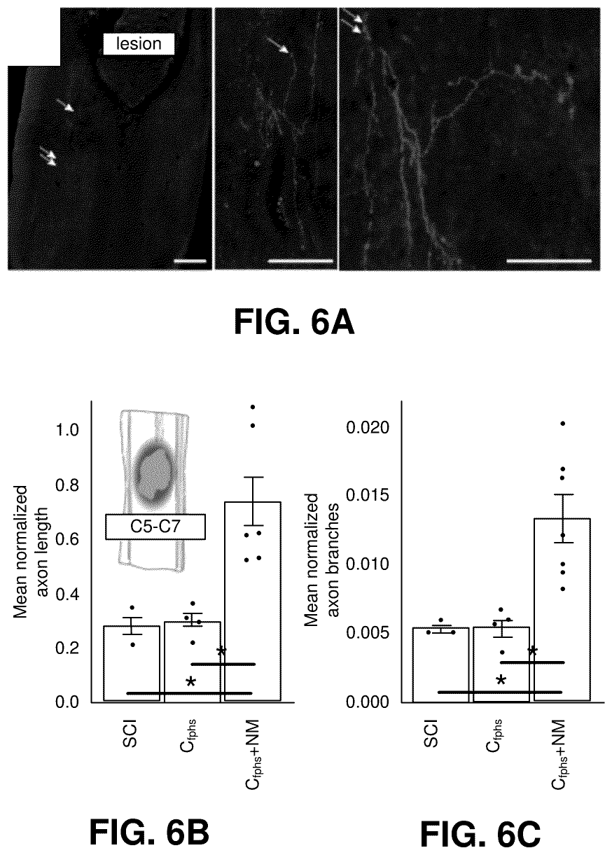

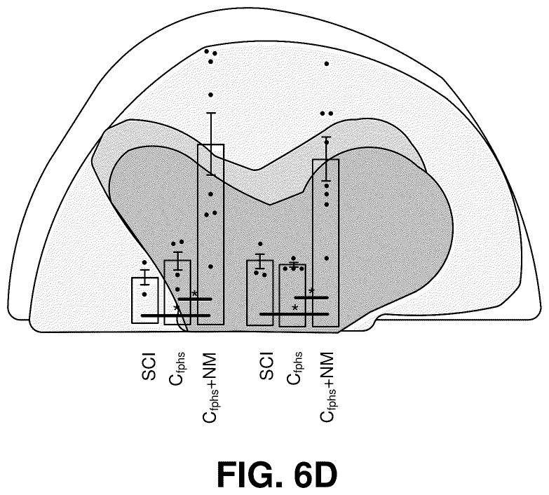

CST axon density and branching caudal to SCI: C4 cervical SCI profoundly reduces CST projections into the cervical enlargement. To assess the efficacy of C_{fphs}, alone and in combination with neuromodulation, in reinnervation by the CST within this territory, a broad search was performed to acquire high-resolution confocal microscope images of most of the labeled CST axons in the enlargement segments (C5-C7) (FIG. 6A). To ensure unbiased and adequate sampling, since CST labeling is sparse, for each rat images of CST axons was acquired from a total area of 5.2 mm²±0.32 mm² (KW=0.3; no difference between groups). The data was randomly down sampled to 15,000 m per rat. In this way, each animal had the same total area sampled and total axon length. Because of this sampling strategy, mean total axon length within a defined ROI (i.e., density) could not be used as a dependent measure. Instead, the mean length of each measured CST axon segment was calculated. To determine if there were group differences in CST axon length, axon branching, and the dorso-ventral axon position (i.e., dorsal horn or intermediate zone/ventral horn) the length of each axon was compared and differences between the groups were found (FIG. 6B; KW=11.30, P=0.0002). NM alone is known to result in 12.5% increase in axon length (see Exp Neurol. 2017 November; 297: 179-189). In stark contrast, the combined C_{fphs}+NM showed a two-fold increased axon length compared to SCI-only (P=0.003) and C_{fphs} (P=0.008). There was no difference between SCI-only and C_{fphs} (P=0.67).

TABLE 1
Normalized mean normalized axon length

| | Normalized increase |
|---|---|
| Treatment | mean axon length |
| SCI | 1.0 |
| NM | 1.125 |
| C_{fphs} | 1.0 |
| C_{fphs} + NM | 2.4 |

A similar effect was observed for axon branch points (nodes; FIG. 6C; KW=11.12, P=0.003). The number of branch points was increased 2.4-fold in the C_{fphs}+NM group compared to SCI (P=0.007) and C_{fphs} groups (P=0.004). A determination was made concerning whether labeling was within either the dorsal or ventral regions (FIG. 6C). There were group differences in dorsal (KW=9.14, P=0.002) and ventral (KW=9.78, P=0.0017; same animal numbers) regions. In both regions, C_{fphs}+NM after SCI (right side of figure) resulted in significantly greater CST outgrowth to the cervical enlargement gray matter than SCI-only (dorsal, P=0.005; ventral, P=0.008) and C_{fphs} only (dorsal, P=0.036; ventral, P=0.006). There was no difference between SCI-only and C_{fphs} (dorsal, P=0.42; ventral, P=0.88). FIG. 6D, the density of CST axons separated by zones in the dorsal and intermediate regions. The plots are overlaid onto a schematic of the spinal cord with contours to aid reference of the relative dorsoventral distribution. These findings show that neuromodulation more than doubled CST length and branching compared with either SCI-only or SCI+C_{fphs}, within the normal territory of CST terminations.

### Discussion

Combinatorial approaches for SCI treatment benefit from incorporation of implantable ‘bio-scaffolds’ to substitute for the extracellular matrix (ECM), to recreate an environment that permits cell adhesion, migration, and axon growth. This underlies the capacity for C_{fphs}, when injected acutely after SCI, to prevent cavity formation chronically, by promoting cell infiltration and tissue adherence, which contributes to reestablishing the ECM. In turn, this substrate acts as a scaffold for axon growth into the lesion. C_{fphs} tissue repair enables significantly reduced axon dieback at the rostral pole of the lesion, thereby maintaining a more caudal location of the CST at the lesion site, both in the dorsal column and terminations in adjoining gray matter. This additional length of axotomized CST axons is leveraged with neuromodulation to promote activity-dependent gray matter sprouting. Activity dependent sprouting where reduced CST axon dieback is observed points to synergistic interactions with C_{fphs}, as neuromodulation has a greater spared axon substrate from which to promote sprouting. Neuromodulation also promoted sprouting at the caudal border of the lesion and farther caudally into the cervical enlargement. Together, these findings show that the novel biomaterial-neuromodulation combinatorial strategy achieves significant lesion repair and promotes CST projections above, at, and below the injury.

C_{fphs} modulates inflammation at the injury site: A key goal for SCI repair is to modulate the inflammatory environment to promote tissue restoration. Indeed, one of major deleterious events after SCI is the uncontrolled inflammation that spreads into neighboring segments creating further damage. Tissue repair mediated by C_{fphs} reduces pro-inflammatory and promotes anti-inflammatory macrophage markers for long-term inflammation resolution. Anti-inflammatory macrophage subtypes migrate and adhere efficiently on a C_{fphs} substrate, while inflammatory macrophages rarely invade a C_{fphs} implant. As the process of cellular infiltration and axon growth unfolds, C_{fphs} is progressively degraded, creating space for the newly forming tissue. Without wishing to be bound to any particular theory, C_{fphs} is believed to limit the spread of secondary damage in adjacent segments and enables effective and persistent cavity mitigation. Maintenance of infiltrated cells and axons at the remodeled lesion site until the chronic post-injury period may be supported by reestablishment of functional vasculature after C_{fphs} injection.

C_{fphs} enables axon growth and maintenance within the remodeled tissue: The results using PDGFRB suggest axons are able to grow into the injury site and survive into the chronic period after early/acute C_{fphs} injection. C_{fphs} supports extensive growth from axotomized and spared axons, identified using Tuj1 staining. Many of these axons are located alongside astrocyte processes oriented toward the lesion epicenter (see FIG. 2E), which normally would not be expected to provide this support. Pericytes and fibroblasts are marked by PDGFRB and, as such, identify fibrous tissue established after SCI that is not permissive to axon growth. In SCI-only animals, CST axons fail to penetrate beyond the interface with high PDGFRB staining. However, in the C_{fphs} groups we consistently observed CST axons farther into these presumably inhibitory territories. These findings suggests that C_{fphs} enables spontaneous, injury-dependent, axon growth into the newly-formed tissue at the injury site. Further experiments are needed to elucidate the mechanism by which C_{fphs} creates a more permissive environment for axon extension.

Rostral injury site remodeling reduces CST axon dieback and local activity-dependent sprouting: With the aid of robust Adeno-Associated Virus (AAV) tracing of CST axons, C_{fphs} is found to substantially reduce CST axon dieback. Referenced to peak GFAP staining, a ˜1000 m caudal shift is observed in surviving CST axons. In C_{fphs} treated animals, CST axons ‘break through’ the lesion margin compared with SCI only (25.1% of axons present at peak GFAP staining, compared with 0.3% for SCI only; FIG. 4E). This distance is approximately 4-fold greater than observed following genetic deletion of Semaphorin pathways in mice. Reduced axon dieback at the lesion site suggests greater survival capacity of axotomized axons. At the site of reduced dieback, the data show that neuromodulation strongly promotes sprouting of CST collateral axon branches into the adjoining gray matter. The combined biomaterial-neuromodulation strategy may recruit complementary mechanisms that enable injury-dependent axon growth by modulating inflammation and an activity-dependent boost in the intrinsic growth state to support gray matter sprouting.

C_{fphs} and neuromodulation cooperate at the lesion site for enhanced CST gray matter projections after SCI: At C3, gray matter CST axon density was the same in the SCI-only and C_{fphs} groups. By contrast, a strong neuromodulation effect on CST axon density was observed. This reflects activity-dependent sprouting at this rostral location, given that neuromodulation drives CST sprouting in uninjured rats and rats with cervical contusion by upregulation of mTOR and Jak/Stat signaling and downregulating PTEN protein expression. At the rostral lesion margin at C4, there is a substantial loss of CST axons in the gray matter in the SCI group. This reduction likely reflects the extensive dieback of CST axons in the dorsal column, and associated loss of collateral branches in the adjoining gray matter. With C_{fphs}, there is maintenance of gray matter axon density at the values observed at C3, with a clear trend to an increase (p=0.08). Like with reduced dieback, gray matter axon maintenance may be enabled by reduced inflammation and tissue restoration at the initial lesion impact zone. Importantly, at this level C_{fphs} combined with neuromodulation shows cooperativity, with a large and significant increase in CST density.

In addition to reflecting improved axon survival, the added significance of reduced CST dieback is that connectivity into the upper cervical gray matter can be enhanced with neuromodulation. Although rostral to the lesion, sprouting at this level can have a beneficial effect. The C3-C4 propriospinal neurons are present at this level and their axons descend within the ventral and lateral gray matter, which should be largely spared after midline dorsal contusion. These neurons comprise a spinal bypass circuit between motor cortex and segmental motor circuits: receiving input from the intact CST, they synapse on cervical motoneurons. At the caudal lesion margin in the cervical enlargement, the effects of C_{fphs} alone and with neuromodulation have a pattern similar to that at C3; neuromodulation provides a boost that is proportional to observed at C3, but the length values are a small fraction of the rostral length.

Combinatorial strategy to promote descending projections rostral and caudal to cervical SCI: This disclosure combines a biomaterial scaffold with a neuromodulation strategy to combat multiple distinct mechanisms underpinning SCI pathophysiology—tissue remodeling at the injury site and motor circuit structural plasticity. The disclosure provides a proof of principle for the use of the combinatorial strategy to improve CST projections together with robust tissue repair after SCI. C_{fphs} injection can be performed during early post-injury surgical procedures and neuromodulation can be entirely non-invasive using magnetic instead of electrical cortical stimulation.

The CST is key to skilled movement control. Individually, C_{fphs} alone improves hind leg gait and posture after both thoracic dorsal hemisection and thoracic contusion (assayed using BBB). Dual motor cortex-spinal cord neuromodulation alone improves guided forelimb locomotion after cervical contusion (horizontal ladder walking). This suggests that the two approaches complement each other because they predominantly target different pathophysiological mechanisms. Further, the present findings suggest that the combined biomaterial neuromodulation approach promote CST projections rostrally and caudally, which would contribute to improving motor recovery. First, C_{fphs} reduces CST axon dieback resulting in a more caudal presence of CST axons at the rostral lesion margin. Second, neuromodulation is able to promote a greater amount of CST sprouting at the rostral pole than C_{fphs} alone, enabled by the reduced axon dieback in the dorsal column. Third, neuromodulation also promotes CST axon sprouting and branching caudally. An increase of approximately 2.5 times was observed with the combined approach. This is substantially greater than the 12% increase observed caudal to cervical contusion injury but without C_{fphs} in a prior study (Zareen et al., 2017, Exp. Neurol. 297, 179-189). Considering C_{fphs} modulation of lesion inflammation and gliosis and neuromodulation-induced CST sprouting, the combined strategy may also restrict caudal encroachment of the lesion into the enlargement segments.

### Methods

Subjects and timeline: Sprague-Dawley female rats between 250 and 275 g body weight were obtained from a certified vendor (Charles River Lab, New York, USA) for this study, which was approved by the CUNY Advanced Science Research Center IACUC. The animals were randomly assigned to one of three groups: SCI only (SCI, N=5); SCI+biomaterial (C_{fphs}, N=5); and SCI+biomaterial+neuromodulation (C_{fphs}+NM, N=7). The study timeline is shown in FIG. 1.

General Surgical procedures: All surgical procedures were conducted under aseptic conditions. Animals were anesthetized with isoflurane (4.0% induction; 1.5-3.0% maintenance). For pain management, we administered analgesics (buprenorphine, 0.03 mg/kg) during surgery, the following day, and longer if needed. Fluids (Ringer's or saline, 10 mL s.c.) were administered after each procedure.

Craniotomy over MCX and headcap: Animals were placed in a stereotaxic frame following induction of anesthesia. A midline scalp incision was made from the frontal to the temporal poles and the periosteum was cleared. Small bilateral craniotomies (approx. 4.5×4.0 mm) were made over the frontal and rostral parietal lobes to expose the forelimb region of primary motor cortex. For each craniotomy, a headcap was formed with dental acrylic cement, anchored to skull screws, to seal the opening and secure the epidural electrical stimulation electrodes (see below). We have successfully used this approach to fit the headcap with the electrode and connectors for electrical stimulation chronically (>10 weeks).

Anterograde tracing of CST from MCX: AAV-2 anterograde tracers were injected in motor cortex to label the corticospinal projection within the caudal forelimb motor representation, as in our previous studies (Yang and Martin, 2023, Brain Stimul. 16, 759-771). Typically, EYFP or mCherry fluorescent AAV tracers (AAV2-CaMKIIa-mCherry and AAV2-CaMKIIa-EYFP) were injected to separately label each hemisphere. Within the craniotomy, a small incision was made in the dura to allow intracortical placement of a micropipette for AAV injection. The micropipette was preloaded with AAV and lowered to a depth of 1.5 mm within the cortex. Next, a small quantity of tracer (300 nL) was injected with a micropump (UMP3, World Precision Instruments) programmed at a rate of 3 nL/s. An additional 3 min was added to maximize diffusion away from the tip. AAV tracers were injected at 4 sites within the forelimb region (AP and ML respective to bregma, 3.0, 3.0; 2.0, 2.5; 2.0, 3.5; 1.0, 3.0 mm). After the injections, the craniotomy was sealed with an acrylic headcap (described above). Injection sites were confirmed histologically at the termination of the experiment.

Cortical epidural electrodes and implantation: We have developed a procedure to activate motor cortex in awake animals daily with electrical stimulation using chronically implanted epidural electrodes (Zareen et al., 2017, Exp. Neurol. 297, 179-189). The bipolar electrode was made from a pair of insulated stainless steel wires (PTechnologies) that were deinsulated on the side contacting the dura (2.0-3.0 mm; 1.5-2.0 mm electrode wire separation). Electrodes were implanted bilaterally over the M1 forelimb areas (with respect to bregma: AP 1.5-2.0; ML±3.0 to 3.5 mm). We verified that stimulation through each electrode evoked a contralateral forelimb movement (or muscle contraction) and not whisker or hindlimb movements, indicating correct placement over the forelimb motor representation. For each hemisphere, the electrode and connector pedestal were secured in an acrylic headcap to easily attach cables to deliver electrical stimulation from an isolated stimulation unit.

Cervical laminectomy and C4 contusion injury: Following anesthesia induction (described above), a midline skin incision was made from the C2 and T2 vertebrae. C4 and adjacent laminae were exposed. The dorsal lamina at C4 was removed to expose both sides of the spinal cord. We used a C4 bilateral contusion SCI using a force-controlled device (Infinite Horizons, PSI). The C4 level was chosen because it is rostral to the motor neuron pools of the cervical enlargement. In this way, the primary injury disconnects the majority of the CST axons in the dorsal column from their neuronal targets, while sparing motoneurons within the cervical enlargement and caudally. After performing the laminectomy, the rat was placed on the platform of the impactor device. The platform was placed on an X-Y moveable stage to align the impact probe at midline above the laminectomy at C4. The impact probe (2.5 mm tip diameter; half-rounded chamfer shape) was programmed to deliver the hit (200 kdyn), which produces a moderate bilateral contusion (Zareen et al., 2017, Exp. Neurol. 297, 179-189). The rat was removed from the stabilization platform and the muscles were reapposed and sutured in layers. The wound was treated with topical antibiotic ointment. Fluid therapy (10 mL lactated Ringers, s.c.) and analgesia (buprenorphine 0.03 mg/kg, s.c.) were administered.

C_{fphs} injection into the C4 contusion site: Chitosan (polysaccharide of N-acetyl-D-glucosamine) hydrogel formulation was prepared and characterized, as previously described (Chedly et al., 2017, Biomaterials 138, 91-107). On day 3 following SCI, under aseptic conditions we re-exposed the dural surface, pierced it, and injected the C_{fphs} intraspinally. In other embodiments, the C_{fphs} is injected within 60 days, within 56 days, within 30 days, within 20 days, within 10 days or within 5 days of injury. Prior to the time of injection, under aseptic conditions the initial suspension was centrifuged and the C_{fphs} pellet was collected. For C_{fphs} injection, the contusion site and lesion epicenter were visualized with the aid of a surgical microscope. Using a mechanical pipette specialized for viscous material (Microman, Gilson), the tip was inserted through a small slit in the dura that avoided the major dorsal spinal vessels at the midline and 2 μL of C_{fphs} were slowly pressure injected into lesion site.

Combined motor cortex-trans-spinal direct current neuromodulation: We used the same neuromodulation protocols as in our previous SCI studies (Yang et al., 2019, Exp. Neurol. 320, 112962; Zareen et al., Exp. Neurol. 307, 133-144). The protocol includes patterned phasic stimulation to the cortex (iTBS) and cathodal direct current to the spinal cord (tsDCS) delivered at the same time and is referred to as intersectional neuromodulation. The neuromodulation therapy is administered in freely moving animals for 28 min/day for 10 sequential days. The protocol is well tolerated with brief signs of redness from the cutaneous trans-spinal electrodes.

Cortical intermittent theta burst stimulation (iTBS): The iTBS pattern consisted of a burst of 3 pulses with an interstimulus interval of 50 ms, repeated 10 times for 2 s, followed by 8 s of no stimulation. This was repeated 20 times for a total of 600 pulses, which is referred to as a “block.” The stimulation protocol included 5 blocks of iTBS over a period of 27 min for a total of 3000 pulses each day. This specific protocol was modelled after human transcranial magnetic stimulation (TMS) studies, showing its efficacy in enabling long-term potentiation. The intensity of iTBS was calibrated each day to deliver 75% of the motor threshold determined by the minimum intensity for a brief train of repetitive stimulation (14-pulse) to produce an overt movement consistently while the animal is held gently and its forelimbs free to move in response to stimulation.

Transcutaneous spinal direct current stimulation (tsDCS): tsDCS was administered using cutaneous fabric hydrogel electrodes (PALS, North Coast Medical). The electrodes first were covered with conductive gel to enhance current flow and reduce skin irritation during the stimulation, and then securely placed onto the skin. One electrode was placed on the dorsal neck spanning C4-T1 (1.2×4.0 cm) and the other was placed on the chest manubrium (2.0×2.0 cm). The dorsal neck electrode is referenced as the cathode. This montage was selected based on results from Finite Element Modelling in the rat, which predicted the area of the spinal cord that receives maximal current flow within the cervical enlargement (Zareen et al., 2017, Exp. Neurol. 297, 179-189). The animal was fitted with a jacket (Braintree Scientific) that covered the electrodes to prevent electrode movement and prevent the wire leads from pulling on the electrodes. Electric current (DC) was delivered using a programmable waveform generator (A-M Systems, model 3800) and stimulus isolation units (A-M Systems, model 3820). tsDCS intensity was ramped up from 0 to 1.5 mA (3×103 mA/mm²) over a 2.5 s period, and after 27 mins of stimulation was ramped back to 0 over 2.5 s.

Histology and immunohistochemistry: After survival times of 8 weeks post-lesion, animals were deeply anesthetized with ketamine and xylazine (70 mg/kg; 6 mg/kg IM), and transcardially perfused with saline and 4% paraformaldehyde (PFA, Sigma) in 0.1 M phosphate buffer. Spinal cords were dissected, postfixed in the same PFA solution, cryoprotected by soaking in 30% sucrose at 4° C. Tissue sections (40 μm) were cut on a cryostat (horizontal spinal cord sections; Leica CM3050 S) or on a sliding microtome and mounted directly on Superfrost slides (VWR).

Immunohistology: Spinal cord sections were first permeabilized by incubation in 0.3% Triton X-100/PBS during 5 min at room temperature and blocked for 1 h in 10% BSA (bovine serum albumin)/PBS, before incubation overnight at room temperature with primary antibodies diluted in 5% BSA/PBS. After washing in PBS (3×5 min), sections were reacted for at least 1 h with appropriate Alexa-405 (1:500; Jackson Labs), or Cy5 (1:1000; Jackson Labs)-coupled secondary antibodies, and DAPI for nuclear staining. After careful rinsing, sections were finally unfolded, dried and mounted in Mowiol (Sigma-Aldrich). Photomicrographs were taken on Leica TCS SP5, Zeiss Micro Apotome, Zeiss 880 confocal, and Keyence microscopes to create a high-resolution montage of the entire horizontal section. These sections were used for assessment of lesion size and GFAP staining, as well as the location of CST labeling, for subsequent confocal imaging. For quantitative analysis of CST axon, images were taken on a Zeiss 880 confocal microscope. The following primary antibodies were used: rabbit-α-glial fibrillary acidic protein, commonly used to reveal astrocytes (GFAP; Dako, 1:2000); mouse anti-B3 tubulin, Tuj-1 (neuron-specific; Covance, 1:1500); Anti-Platelet derived growth factor receptor-beta (PDGFRB; Abcam (ab32570; 1:500).

Lesion cavitation and scarring: The lesion epicenter was used to measure cavity area defined as cell-free zones devoid of DAPI staining. GFAP staining was measured as a region of interest (ROI; 100 m wide band) surrounding the cavity or restored cavity-tissue border expressed as an optical intensity multiple in a similar region at the rostral end of the section (˜2 mm rostral to SCI).

CST axon dieback: The number of CST axons labeled with fluorescent AAV tracers in the dorsal columns rostral to the injury were analyzed to quantify dieback. We measured the rostral dieback in proximity to the lesion margin determined by the peak GFAP signal (see FIGS. 4A-C) (referenced as 0.0 mm) in the same section). Counting bins were marked by a gridline placed every 300 m from the lesion margin. An axon that crossed a gridline was counted and tallied for each bin in one section. Individual differences in tracer efficacy were accounted for by normalizing to the plateau number of axon crossings, averaged from 2 bins (300 m apart), which was typically >1.5 mm rostral to the lesion margin.

CST gray matter axon analyses: For CST-labeled axons at and rostral to SCI (FIGS. 5A-5C), we measured the total length within a region of interest (ROI) (C3: 300 μm2; rostral lesion margin 300×600 μm2). The total axon length within the ROI was used to obtain CST axon density. The measurement was normalized for tracer efficacy (see next section). For FIGS. 6A-6D, due to the sparseness of CST axon labeling caudal to SCI (C5-C7), we estimated length using a two-step method. First, we carefully screened for the presence of labeled axons using the fluorescence microscope, which is interfaced with a Zeiss 880 confocal microscope, at low magnification. We confirmed the presence of labeling using confocal microscopy, whereupon we scanned and saved images (as z-stacks) for subsequent length measurements. For all groups, we essentially imaged all sections where labeling was present. Second, we used NeuroLucida (MBF Bioscience) and measured the lengths of all axons at 20× magnification. Screening for axons, confocal image acquisition, and axon measurements were conducted by lab personnel blinded to animal group. To eliminate any group sampling biases for total length of axons measured, we randomly down-sampled all axons measurement to 15,000 m of axons for each animal. We computed the mean (±SEM) length of all measured axons. To estimate group effects on axon branching, we marked all axon nodal (branch) points. To estimate depth distribution, each axon measurement was assigned a depth based on serial section order and a fiducial mark (ventral border of the dorsal column). In this way we were able to place each axon into either the dorsal horn or the intermediate zone/ventral horn. To summarize, each axon was associated with a measured length, number of nodes, and gray matter depth.

To test whether there were group differences in the region immediately caudal to the lesion, we subjected the C5 axon measurements to a different analysis. For each animal, we tallied up the total C5 axon length and divided by the image area to compute a measure of axon density in the same way as the rostral length analyses. Instead of using a fixed ROI, the ROI could vary to capture axons and exclude sampling areas devoid of axons, and we ensured the total ROI area was the same across groups.

CST tracer efficacy normalization: Images of axons labeled with fluorescent AAV tracers were obtained with high-resolution confocal or apotome microscopy. Axon length was measured from horizontal sections using NeuroLucida (MBF Bioscience). To control for variability in axon length and density measurements due to the efficacy of tracer uptake, we used the total labeled axon count method rostral to injury. Since all axons in the dorsal column were axotomized by the contusion injury, we counted the number of labeled axons in the lateral and ventral columns at C2, which represents the maximal spared source of axons caudal to the injury. We corrected for tracer efficacy by dividing the measured axon length using NeuroLucida by the number of C2 axons in the lateral and ventral columns. Confocal zstacks captured 3 slices spanning a small range (10-15 um centered from the middle of the section) to minimize artifacts. A mask was drawn at the border between the white and gray matter. All axons in the white matter (visible in at least ⅔ slices in the stack), excluding the dorsal columns, were counted. Comparison between two scorers (one blinded) showed a high intraclass correlation coefficient (ICC=0.97; 95% C.I.=0.88-0.99).

Statistics: Kruskal-Wallis non-parametric tests were used to compare group differences with small and uneven sample sizes (KW; a 0.05). Post hoc comparisons were corrected for false-discovery with the Benjamini, Krieger and Yekutieli two-stage step up method. Data are reported as means±SEM.

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio. For example, a composition can include a pharmaceutically acceptable ester or amide of a compound herein. In certain implementations, a composition includes a pharmaceutically acceptable salt of a compound herein. Non-limiting examples of pharmaceutically acceptable salts include carboxylate salts, amino acid addition salts and zwitterionic forms thereof, which are known to those skilled in the art as suitable for use with humans and animals. In cases where a compound is sufficiently basic or acidic to form a stable nontoxic acid or base salt, a composition includes a pharmaceutically acceptable salt of the compound. Non-limiting examples of pharmaceutically acceptable salts are organic acid addition salts formed with acids that form a physiological acceptable anion, non-limiting examples of which include tosylate, methanesulfonate, acetate, citrate, malonate, tartarate, succinate, benzoate, ascorbate, alpha-ketoglutarate, and alpha-glycerophosphate. Suitable inorganic salts may also be formed, including hydrochloride, sulfate, nitrate, bicarbonate, and carbonate salts. Pharmaceutically acceptable salts are obtained using standard procedures known in the art. For example, pharmaceutically acceptable salts may be obtained by reacting a sufficiently basic compound with a suitable acid affording a physiologically acceptable anion. Alkali metal (for example, sodium, potassium or lithium) or alkaline earth metal (for example, calcium, magnesium) salts of carboxylic acids and other anionic groups in molecules within a pharmaceutical composition also are contemplated.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for treating a neurological disorder in an animal, the method comprising steps of:
administering chitosan Fragmented Physical Hydrogel Suspension (C_{fphs}) to the animal at a site of spinal cord injury (SCI);
performing both (1) cerebral cortex neuromodulation and (2) spinal cord direct current stimulation (tsDCS) on the animal for at least 10 minutes per day; and
repeating the step of performing for at least 10 days.

2. The method as recited in claim 1, wherein the step of performing is performed for at least 20 minutes per day.

3. The method as recited in claim 1, wherein the step of performing is performed for at least 28 minutes per day.

4. The method as recited in claim 1, wherein the step of performing is performed for less than 1 hour per day.

5. The method as recited in claim 1, wherein the at least 10 days are 10 sequential days.

6. The method as recited in claim 1, wherein the cerebral cortex neuromodulation is motor cortex (MCX) neuromodulation.

7. The method as recited in claim 1, wherein the step of administering is performed within 56 days of an injury to the site of spinal cord injury (SCI).

8. The method as recited in claim 1, wherein the step of administering is performed within 5 days of an injury to the site of spinal cord injury (SCI).

9. A method for treating a neurological disorder in an animal, the method comprising steps of:
administering chitosan Fragmented Physical Hydrogel Suspension (C_{fphs}) to the animal at a site of spinal cord injury (SCI), wherein the C_{fphs} comprises chitosan hydrogel microparticles with a median size d50, obtained from a number distribution, of from 1 to 500 μm, a degree of acetylation of less than or equal to 20%, wherein chitosan in the hydrogel microparticles is from 0.25 to 5% concentration by weight, based on the total weight of the C_{fphs};
performing both (1) motor cortex (MCX) neuromodulation and (2) spinal cord direct current stimulation (tsDCS) on the animal for at least 10 minutes per day; and
repeating the step of performing for at least 10 days.

10. The method as recited in claim 9, wherein the step of performing is performed for at least 20 minutes per day.

11. The method as recited in claim 9, wherein the step of performing is performed for at least 28 minutes per day.

12. The method as recited in claim 9, wherein the step of performing is performed for less than 1 hour per day.

13. The method as recited in claim 9, wherein the at least 10 days are 10 sequential days.

14. The method as recited in claim 9, wherein the step of administering is performed within 56 days of an injury to the site of spinal cord injury (SCI).

15. The method as recited in claim 9, wherein the step of administering is performed within 5 days of an injury to the site of spinal cord injury (SCI).
